(19) United States
(12) Patent Application Publication
(10) Pub. No.: US 2024/0350061 A1
(43) Pub. Date: Oct. 24, 2024

(54) STRETCHABLE SENSOR ARRAY AND APPLICATIONS THEREOF

(71) Applicant: TAO TREASURES LLC DBA NANOBIOFAB, Frederick, MD (US)

(72) Inventors: Xiaonao LIU, Frederick, MD (US); Taejun KO, Clarksburg, MD (US); Derese Getnet, Baltimore, MD (US)

(21) Appl. No.: 18/645,318

(22) Filed: Apr. 24, 2024

Related U.S. Application Data
(60) Provisional application No. 63/498,001, filed on Apr. 24, 2023.
(60) Provisional application No. 63/498,325, filed on Apr. 26, 2023.

(51) Int. Cl. A61B 5/268; A61B 5/00; G09B 23/28; G09B 23/30

(52) U.S. Cl. CPC A61B 5/268; A61B 5/0022; A61B 5/447; G09B 23/281; G09B 23/285; G09B 23/288; G09B 23/30; A61B 2562/043; A61B 2562/164

(57) ABSTRACT

A stretchable sensor array system includes a stretchable substrate, a sensor array comprising a plurality of sensors deposited on the substrate, and a computing device. Each senor has a conductive electrode that contains one or more carbonaceous materials and is configured to respond to a parameter selecting from pressure, temperature, humidity, and chemical substances, and each sensor is signally connected to the computing device and transmits signals representing the parameter to the computing device for storage and/or processing. The stretchable sensor array system can be used for patient monitoring such as pressuring ulcer monitoring and prevention as well as training simulation such as palpation training and analysis. A training simulator includes one or more stretchable sensor arrays affixed to a manikin, a garment, or a helmet, etc., and provide data reflecting the training activity, e.g., palpation.

### CROSS-REFERENCE TO RELATED APPLICATIONS

This application claims the benefit of priority under 35 U.S.C. § 119 to U.S. Provisional Application No. 63/498,001, filed Apr. 24, 2023, and U.S. Provisional Application No. 63/498,325, filed on Apr. 26, 2023, the entire contents of which are incorporated herein by reference in their entireties.

### TECHNICAL FIELD

The present invention generally relates to flexible and stretchable sensor array and applications hereof, more specifically related to sensor array and its application in the field of healthcare, including pressure ulcer prevention and monitoring, and medical simulation and training.

### BACKGROUND

Smart sensors are widely used in patient monitoring in the healthcare industry. Existing smart sensor technologies, which utilize arrays of rigid sensors embedded in flexible media or stretchable circuits, suffer from several drawbacks. These include high cost, limited data collection due to discrete sensing units and dead zones, and rigidity-induced artifacts in simulated tissue properties.

For example, pressure ulcers, also known as bedsores or decubitus ulcers, are localized injuries to the skin and/or underlying tissue, typically over bony prominences. They are caused by prolonged focused pressure, shear, or friction, and are commonly observed in patients with limited mobility, such as those confined to beds or wheelchairs. Pressure ulcers are painful, lead to complications, and can substantially impact the quality of life for affected individuals.

Various devices and methods have been proposed for pressure ulcer prevention and monitoring. Current methods for pressure ulcer prevention typically involve manual repositioning of the patient at regular intervals to alleviate pressure on vulnerable areas of the body. However, this approach can be labor-intensive, time consuming, and may not effectively address the individual risk factors that contribute to pressure ulcer development. Moreover, some existing devices include pressure-sensing mats, wearable devices, and electronic monitoring systems. However, these devices often lack the flexibility, sensitivity, and specificity required for early detection and prevention of pressure ulcers. Furthermore, such devices may not be able to accommodate complex body shapes and movements, or accurately monitor other relevant factors, such as temperature, humidity, and volatile organic compounds (VOCs).

In another example, medical simulators and manikins play a critical role in the training and education of healthcare professionals by providing a realistic and controlled environment for the development and refinement of essential skills. However, current designs often lack the necessary sensitivity, responsiveness, and durability to accurately simulate real-life situations, provide instantaneous feedback on performance, and withstand repeated use.

There is a need for advanced, cost-effective sensor technologies that overcome these limitations.

### SUMMARY OF THE DISCLOSURE

In one embodiment of the current disclosure, a stretchable sensor array system includes a stretchable substrate, a sensor array comprising a plurality of sensors deposited on the substrate, and a computing device. Each sensor has a conductive electrode that contains one or more conductive materials and is configured to respond to a parameter selected from pressure, temperature, humidity, and chemical substances, and each sensor is signally connected to the computing device and transmits signals representing the parameter to the computing device for storage and/or processing.

According to one aspect of an embodiment, the stretchable sensor array system has a plurality of stretchable substrates, each having a sensor array deposited thereon.

According to another aspect of an embodiment, the substrate is composed of materials selected from the group consisting of polydimethylsiloxane (PDMS), polyurethane, silicone rubber, fluorinated elastomers, butadiene-based elastomers, isoprene-based elastomers, styrene-butadiene rubber, acrylonitrile-butadiene rubber, natural rubber, and synthetic rubbers such as neoprene, nitrile rubber, and ethylene propylene diene monomer (EPDM) rubber, or other suitable stretchable polymers.

According to still another aspect of an embodiment, the conductive material is selected from conductive fabrics, conductive polymers, or conductive nanomaterial composite (carbon nanotubes, graphene, carbon blacks, silver nanowires, copper nanowires, gold nanowires, titanium dioxide, zinc oxide, magnesium oxide, and molybdenum disulfide).

In yet another embodiment of the present disclosure, a method for fabricating a stretchable sensor includes the steps of directly writing sensing units onto a stretchable substrate using a direct writing process. The writing process deposits sensing units containing conductive polymers or conductive nanomaterial composite.

In a further embodiment of the current disclosure, a system for pressure ulcer prevention and monitoring includes a stretchable sensor array system, and more sensor arrays are affixed to a patient's skin or a device the patient is in a physical contact with; and a patient or caregiver notification system operatively connected to the computing device.

According to one aspect of an embodiment, the computing device is a workstation or a handheld device, and a software program is installed on the computer device for receiving, processing, and/or displaying data.

According to one aspect of an embodiment, the system for pressure ulcer prevention and monitoring cloud-based platform for storing, processing, and analyzing data related to factors associated with pressure ulcer development, and for facilitating communication between the sensor array system and the patient or caregiver notification system.

According to still another embodiment, a method for preventing and monitoring pressure ulcers in a patient using the stretchable sensor array system includes the steps of: applying one or more sensor arrays to a patient's skin or a device the patient is in a physical contact with; detecting one or more parameters selected from pressure, temperature, humidity, and volatile organic compounds (VOC) using the one or more sensor array; and transmitting the data to the computing device to be analyzed and to determine one or more conditions of the patient.

In yet another aspect, the disclosure provides a method for preventing and monitoring pressure ulcers using the stretchable sensor array system. The method comprises the steps of: (a) applying the sensor array system to a patient's skin or incorporating it into a wearable device; (b) continuously monitoring pressure, visualization, location, complex body shapes, temperature, humidity, and VOC using the sensing units; (c) transmitting the data to a data processing module; (d) analyzing the data to predict the development of pressure ulcers; and (e) alerting a patient or caregiver when a predetermined threshold is exceeded, so that appropriate intervention measures can be taken.

In another aspect of the embodiment, the device the patient is in contact with is a bandage, a garment, a cushion, a mattress, a chair, a wheelchair, and a mattress topper.

In one aspect of the disclosure, the stretchable sensor system can be customized to fit different body shapes and sizes, allowing for a more personalized and effective monitoring system.

In another aspect of the disclosure, a sensor array is provided, comprising four sensors: a pressure sensor, a temperature sensor, a humidity sensor, a VOC sensor. These sensors are placed above the stretchable substrate and covered by membranes that allow air permeation in some cases. Depending on the size of the body area, each part of the sensor set consists of from 1 to 100 sensors, and each sensor has a size of 10 to 600 mm². The wireless data transfer using communication system allows for remote monitoring and tracking of pressure points, enabling personalized recommendations and adjustments for individual users.

The system and method of the present disclosure can be applied to seat cushions for wheelchairs or chairs, hospital beds, mattress toppers or overlays, and wearable devices, providing a personalized and effective monitoring solution for pressure ulcer prevention.

In a further aspect of the disclosure, a pressure-sensing mattress is provided, comprising a base material selected from the group consisting of foam, gel, air cells, and silicone rubber, and a stretchable pressure sensor comprising at least one material selected from the group consisting of conductive fabrics, conductive polymers, or conductive nanomaterial composite (carbon nanotubes, graphene, carbon blacks, silver nanowires, copper nanowires, gold nanowires, titanium dioxide, zinc oxide, magnesium oxide, and molybdenum disulfide). The pressure-sensing mattress may also include a stretchable temperature sensor comprising at least one material selected from the group consisting of graphene, carbon nanotubes, elastomers, and polymer composites, as well as a stretchable humidity sensor comprising at least one material selected from the group consisting of graphene oxide, metal-organic frameworks (MOFs), and conducting polymer nanofibers.

In one aspect of the disclosure, the temperature sensor comprises a material selected from the group consisting of thermistors, resistance temperature detectors, thermocouples, infrared thermometers, fiber optic sensors, liquid crystal thermometers, bimetallic strips, diode temperature sensors, microelectromechanical systems (MEMS) based temperature sensors, and nanomaterial-based temperature sensors.

The pressure-sensing mattress also comprises a humidity sensor comprising at least one material selected from the group consisting of capacitive sensors such as metal oxide capacitors, polymer capacitors, interdigitated capacitive sensors, resistive sensors such as ceramic humidity sensors, resistive polymer humidity sensors, hygrometers, and nanomaterials such as graphene oxide and carbon nanotubes.

In one aspect of the disclosure, the VOC sensor comprises a material selected from the group consisting of gas chromatography sensors, metal oxide sensors, conducting polymer sensors, surface acoustic wave sensors, quartz crystal microbalance sensors, optical sensors, electrochemical sensors, photoionization detectors, and ion mobility spectrometers.

The pressure-sensing mattress utilizes a wireless data transfer system comprising Bluetooth, Wi-Fi, etc.

The AI-powered analysis system is capable of analyzing overall pressure distribution, patient's body profiles and postures, pressure distribution during patient's movements and position changes, time spent in a particular position, humidity levels, and temperature levels, to predict pressure points that may lead to pressure ulcers.

The pressure-sensing mattress is applicable for use in seat cushions for wheelchairs or chairs, hospital beds, mattress toppers or overlays, and wearable devices.

In one embodiment, the stretchable sensor system utilizes a direct writing process equipped with conductive material cartridges capable of creating flexible and durable sensors. These sensors are directly deposited onto the manikin surface following a predefined layout that corresponds to specific anatomical or design-related pressure points.

The software accompanying the sensor system is designed to process the data collected in real time, using algorithms optimized for recognizing and interpreting pressure variations. It provides outputs in various formats, including heat maps and pressure-time graphs.

In one additional embodiment of the current disclosure, a medical training simulator includes a stretchable sensor array system of, an object that a trainee interacts with, and the sensor array is affixed to the object.

In one aspect of the embodiment, the sensor array includes a plurality of pressure sensors and is configured to detect various palpation techniques, such as light palpation, deep palpation, light ballottement, and deep ballottement, and to produce corresponding data outputs.

In another aspect of the embodiment, the object is a manikin, a garment, a vest, or a helmet.

In still another embodiment of the current disclosure, a method for palpation simulation includes the steps of: affixing the sensor array to the object, the object being a manikin and the sensor array comprises a plurality of pressure sensors; performing palpation on the sensor array and causing the sensor array to transmit sensor data to the computing device; and processing the sensor data and display the processed data, wherein the processed data reflects pressure applied during the palpation.

In another embodiment of the present disclosure, the stretchable sensor array system is applicable in various medical training scenarios. It is useful for simulating surgical procedures like laparoscopy and wound closure, monitoring orthopedic maneuvers, assessing pressures in intravenous catheter insertion and phlebotomy, and providing feedback during CPR chest compression training. Additional applications include palpation simulation during physical exams, pressure monitoring to prevent bedsores, and evaluating force during obstetric simulations like childbirth.

### BRIEF DESCRIPTION OF THE DRAWINGS

FIG. 1 is a schematic illustration of the stretchable sensor array system according to one embodiment of the present disclosure.

FIG. 2 illustrates the scenario in which a sensor array is being used to monitor a patient to prevent pressure ulcers.

FIG. 3A shows the stretchable pressure sensor array affixed on a manikin for abdominal palpation training.

FIG. 3B shows an exemplary stretchable pressure sensor array.

FIG. 3C shows another exemplary stretchable pressure sensor.

FIG. 4A shows the resistance of the sensor changes in response to pressure applied to the sensor.

FIG. 4B shows responses of a pressure sensor to light and deep abdominal palpation.

FIG. 5 summarizes the range and average forces applied during deep and light palpations.

FIG. 6 shows the fabrication process of the stretchable sensor array system.

### DETAILED DESCRIPTION OF THE DISCLOSURE

The following detailed description, in conjunction with the accompanying drawings, provides a more complete understanding of the disclosure and its various embodiments. The description is not intended to be limiting, and modifications and variations within the scope of the disclosure will be apparent to those skilled in the art.

### Embodiment I

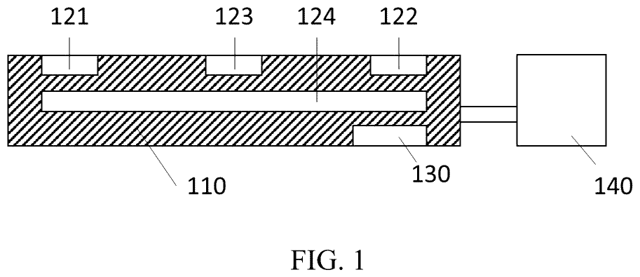

FIG. 1 is a schematic depiction of a monitoring system (100) according to one embodiment of the disclosure. It shows a cross-sectional view of a stretchable sensor array that includes a stretchable substrate (110), a plurality of sensing units (120) disposed on the stretchable substrate (110), and a flexible PCB board (130), and a computing device (140) connected to the substrate (110).

The substrate (110) may be composed of materials such as polydimethylsiloxane (PDMS), polyurethane, silicone rubber, fluorinated elastomers, butadiene-based elastomers, isoprene-based elastomers, styrene-butadiene rubber, acrylonitrile-butadiene rubber, natural rubber, and synthetic rubbers such as neoprene, nitrile rubber, and EPDM rubber, or other suitable stretchable polymers. The substrate (110) provides mechanical support for the sensing units (120) and allows the sensor array system (100) to conform to complex body shapes and movements.

Each sensing unit includes conductive electrodes made from conductive fabrics, conductive polymers, or conductive nanomaterial composite (carbon nanotubes, graphene, carbon blacks, silver nanowires, copper nanowires, gold nanowires, titanium dioxide, zinc oxide, magnesium oxide, and molybdenum disulfide).

The sensor array in FIG. 1 includes four types of sensing units—a temperature sensor (122), a humidity sensor (121), and a VOC sensor (123) at or close to the surface of the substrate, as well as a pressure sensor (124) embedded in the substrate. The temperature sensor, the VOC sensor, and the humidity sensor are each covered by a piece of membrane that allows air permeation. During operation, the temperature sensor and the humidity sensor are placed close to or even in direct contact with or patient's skin for more accurate measurements. The size, number, and distribution of sensing units and the dimension of the substrate (110) can be adjusted according to the particular application. For example, the substrate can be 8′×8′ or 0.1′×0.1′ in size. The size of the sensor can be from 4-600 mm². A substrate may have one sensor or one hundred sensors deposited thereon.

The sensors are connected to the flexible PCB (130), which contains one or more electrical circuits signally connected to the computing device (140). Depending on how the circuitry is designed, it may simply receive and transfer data or also include embedded programs to process data.

The computing device (140) contains a non-volatile memory to store data, e.g., signals from the sensor array, a processor to convert and process data, and a user interface such as a display with a touch screen and/or connected to a keyboard. The computing device (140) is connected to the sensor through electrical wires or wirelessly when the sensor system is properly equipped, e.g., containing circuitry, a power source, and a wireless communication means. For example, the stretchable sensor array system in this embodiment communicates with a computing device through wired connections or wireless communications. The wireless communication can include Bluetooth low energy (BLE) technology for low-power, real-time data transmission between the sensor array system and a remote computing device. This allows for remote monitoring and tracking of pressure points, enabling personalized recommendations and adjustments for individual users.

The computing device (140) can be a computer workstation or a hand-held device.

According to one aspect of this embodiment, the process of making the system (100) starts with selecting a suitable flexible and stretchable substrate (110). Among many factors, the substrate needs to be durable and flexible under extensive and continuous mechanical deformation.

An ink containing an elastomer and conductive nanomaterials is used as the raw material for making the sensor element. The elastomer, the nanomaterials, and other auxiliary ingredients are subjected to a rigorous stirring process to form a homogenous mixture. Other homogenization techniques such as sonication or high-speed mechanical mixing may be employed. The properties of the elastomer and the concentration and type of conductive nanomaterial are strategically chosen based on the desired application, allowing for customization of the sensor's elasticity and pressure sensitivity.

This conductive composite ink is directly written on the substrate by techniques such as inkjet printing, screen printing, aerosol jet printing, material jetting, and liquid deposition. These methods enable precise deposition and patterning of the sensing units.

### Embodiment II

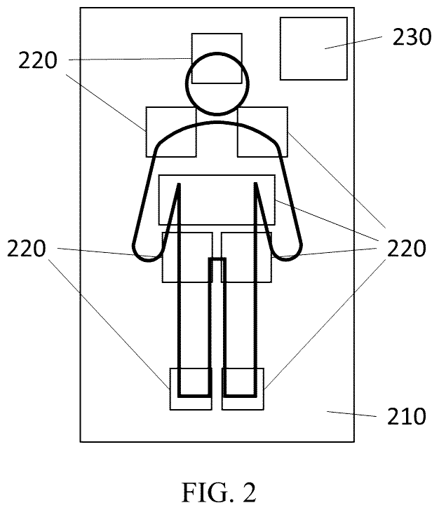

FIG. 2 illustrates an application of the stretchable sensor array system to monitor pressure ulcers. Pressure ulcers, also known as bedsores or decubitus ulcers, are localized injuries to the skin and/or underlying tissue, typically over bony prominences. They are caused by prolonged focused pressure, shear, or friction, and are commonly observed in patients with limited mobility, such as those confined to beds or wheelchairs.

Various devices and methods have been proposed for pressure ulcer prevention and monitoring. Current methods for pressure ulcer prevention typically involve manual repositioning of the patient at regular intervals to alleviate pressure on vulnerable areas of the body. However, this approach can be labor-intensive, time consuming, and may not effectively address the individual risk factors that contribute to pressure ulcer development. Moreover, some existing devices include pressure-sensing mats, wearable devices, and electronic monitoring systems. However, these devices often lack the flexibility, sensitivity, and specificity required for early detection and prevention of pressure ulcers. Furthermore, such devices may not be able to accommodate complex body shapes and movements, or accurately monitor other relevant factors, such as temperature and humidity. Using the sensor arrays according to FIG. 1 to monitor pressure ulcers cures some of the deficiencies in the current technology.

According to Embodiment II, one or more sensor arrays (220) are applied to a patient's skin or embedded in a mattress (210) that the patient lays on. The sensor arrays include pressure sensors, temperature sensors, humidity sensors, and/or VOC sensors that detect volatile organic compounds (VOC) emitting from patient. The VOC data can be correlated with certain conditions, diseases or wounds, as described in detail in Applicants U.S. patents and patent publications, e.g., U.S. Pat. No. 11,353,419 B2 (issued Jun. 7, 2022) and US 2022/0369965 A1 (published Nov. 24, 2022).

The sensors continue to monitor the parameters such as pressure, temperature, humidity, and VOCs, and transmits the data to a computing device (230) embedded in the mattress (210). The computing device (230) has executable software programs installed thereon that enables the collection, analysis, and output of the result to a user. In this case, the software program may set threshold values of certain parameters and output requests to a healthcare professional to take proper measures. For example, if the data indicates prolonged immobility exceeding a threshold value or a pressure (or temperature, humidity) that exceeds a threshold value, the computing device (230) outputs a notification to alter a caregiver on duty or the patient herself through a notification system. The notification system may have an audible alarm, a visual indicator, and/or a tactile indicator.

When the sensor array is installed in the mattress, there is more room to install the circuitry and data transmission devices, e.g., Bluetooth devices, so that the computing device (230) may have more functionalities.

The base material of the mattress can be selected from foam, gel, air cells, and silicone rubber. The stretchable pressure sensor can be one used in Embodiment I. E.g., it may contain at least one material selected from elastomers, conductive polymers, carbon nanotubes, graphene, nanocomposites, conductive fabrics, conductive inks, silver nanowires, copper nanowires, gold nanowires, titanium dioxide, zinc oxide, magnesium oxide, and molybdenum disulfide. In one embodiment. The pressure-sensing mattress may also include a stretchable temperature sensor containing at least one material selected from graphene, carbon nanotubes, elastomers, and polymer composites, as well as a stretchable humidity sensor comprising at least one material selected from graphene oxide, metal-organic frameworks (MOFs), and conducting polymer nanofibers.

In variations of Embodiment II, the sensor array can be installed in any items that the patient may remain in contact with for an extended period of time, including seat cushions for wheelchairs or chairs, hospital beds, mattress toppers, or overlays.

The computing device (230) may employ AI analysis algorithms to analyze data received from the sensor array. For example, the AI algorithm may enable the prediction of pressure points that may lead to pressure ulcers, taking into account factors such as overall pressure distribution, patient's body profiles and postures, pressure distribution during patient's movements and position changes, time spent in a particular position, humidity levels, and temperature levels.

In operation, the stretchable sensor array system of the present disclosure is applied to the surface of a mattress, seat cushion, or wearable device, and continuously monitors factors associated with pressure ulcer development. The data collected by the sensors is transmitted to the data processing module, which analyzes the data and generates alerts or recommendations for patient repositioning or other interventions to prevent pressure ulcers. The alerts or recommendations can be sent to a caregiver, nurse, or the patient themselves through a remote monitoring device.

In a variation of the embodiment, the pressure-sensing mattress may incorporate additional features or functionalities that can be activated by commands based on sensor data and results. For example, if the sensor data indicates prolonged immobility without human intervention, the computing device may elevate the alter or warning to a higher level, or issue commands to provide heating, cooling, vibrations, massage functions, or to change the firmness level of the mattress to temporarily alleviate the condition.

### Embodiment III

Sensor arrays can be used in medical simulation and training, e.g., medical simulators, and manikins to improve the quality, accuracy, and effectiveness of various medical training scenarios. It is useful for simulating surgical procedures like laparoscopy and wound closure, monitoring orthopedic maneuvers, assessing pressures in intravenous catheter insertion and phlebotomy, and providing feedback during CPR chest compression training.

Such sensor arrays may also be applied to full-flight simulators and PARCC test trainers, as well as other potential applications such as detecting suprapubic pressure during challenging childbirth, abdominal palpation training, and sensing pressure on the fetal head during ultrasound.

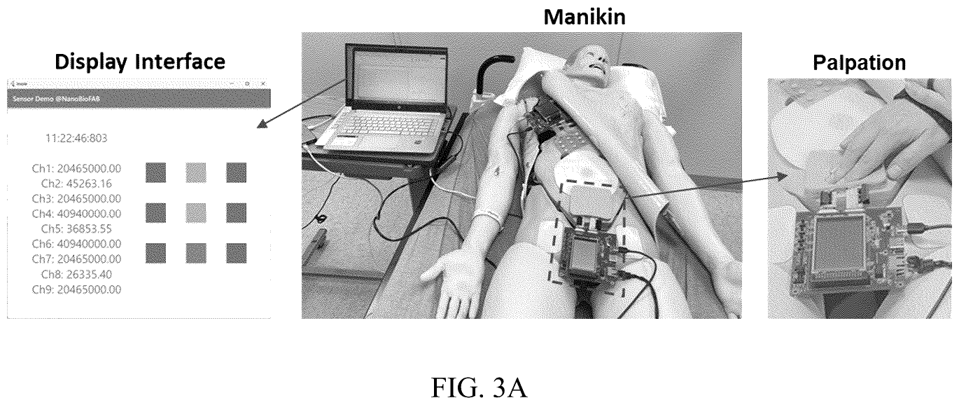

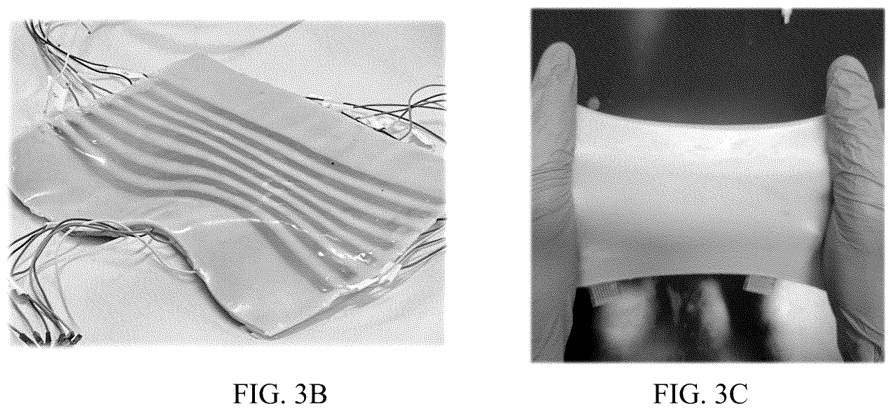

FIG. 3A is a schematic representation of the stretchable pressure sensor pad being applied to a medical simulation manikin, showing the sensor's attachment to the manikin's abdominal surface. The sensor pad is connected to a computer for monitoring and recording the changes in electric signals during abdominal palpation validation test. The sensor pad in FIG. 3A has an array of 3×3 pressure sensors. The display interface of the computer shows 9 squares, corresponding to 9 pressure sensors. When a pressure sensor is pressed, the color of the corresponding square changes. Different colors reflect different magnitudes of applied pressures.

FIG. 3B shows an exemplary stretchable pressure sensor pad. It is 20×20 cm² in size and has 7×7 pressure sensors with associated electrical wires embedded in the substrate. The pressure sensor pad is highly stretchable, as shown in FIG. 3C, which makes it more comfortable to wear and can accommodate a wide range of motion without breaking. During abdominal palpation training, the sensor pad is pressed upon, which changes the resistance of the pressure sensors in the sensor pad. The resistance change is monitored and recorded using the computer and correlated with the position and magnitude of the pressure being applied.

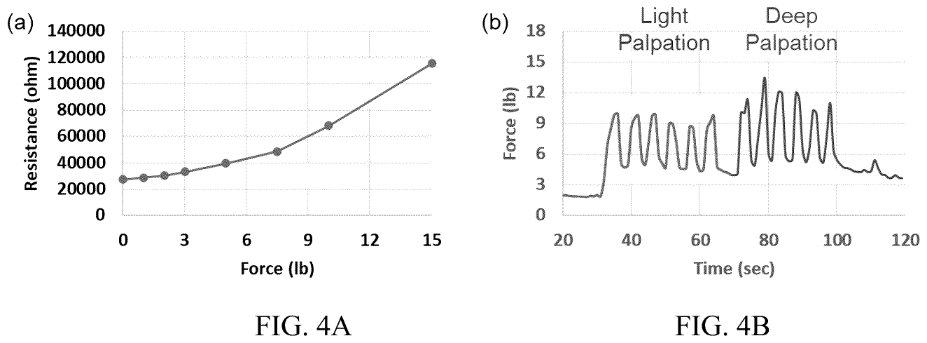

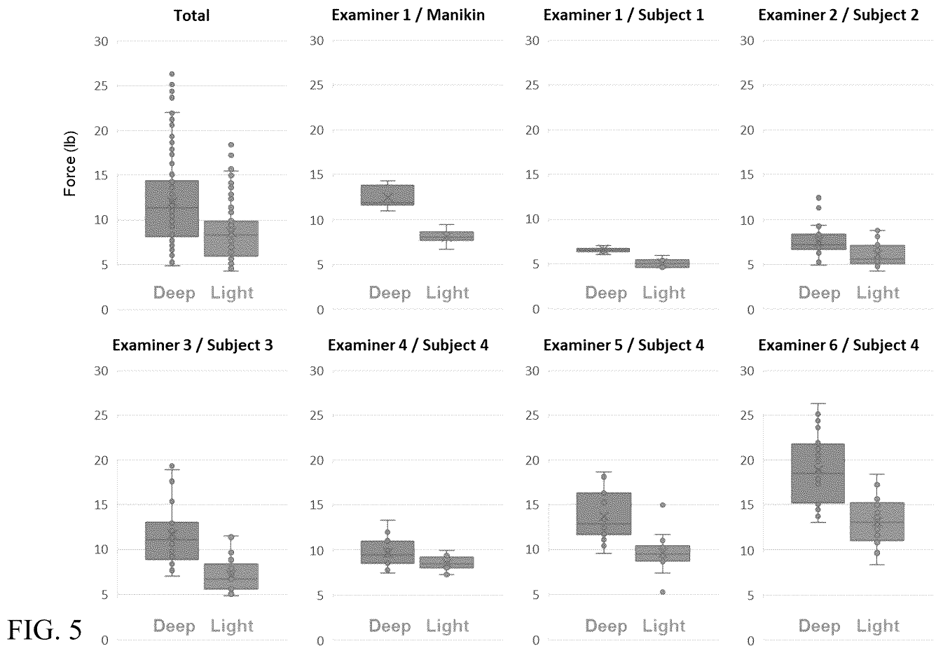

FIG. 4A illustrates the relationship between the applied force and the resistance of the stretchable pressure sensor used for the abdominal palpation test. As the applied force increases, the resistance of the sensor increases, indicating its sensitivity to different force levels.

FIG. 4B shows the sensor's response during abdominal palpation, which includes six light palpations followed by six deep palpations. It demonstrates the ability of the stretchable pressure sensor pad to accurately detect and differentiate between the light and deep palpations.

FIG. 5 is a box chart summarizing the results of the validation tests performed on the stretchable pressure sensor, indicating the range and average forces applied during deep and light palpations. The chart highlights the consistency of the sensor's readings and its potential for use in medical simulators and training devices.

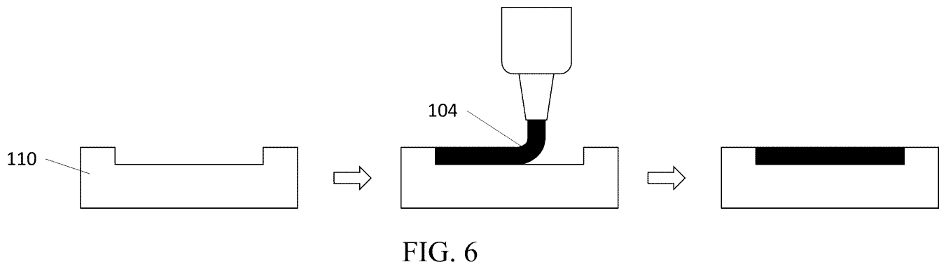

FIG. 6 is a schematic illustration of the fabrication process for the flexible, stretchable sensor, including the direct writing of the sensing material (104) onto a flexible, and stretchable substrate (110). The direct writing techniques can include inkjet printing, aerosol jet printing, screen printing, material jetting, and liquid deposition. The conductive ink can be prepared with materials such as carbon nanotubes, graphene, carbon blacks, silver nanowires, copper nanowires, gold nanowires, titanium dioxide, zinc oxide, magnesium oxide, and molybdenum disulfide.

The present disclosure is not limited to the specific embodiments, materials, and configurations described herein. Various modifications and adaptations of the disclosure can be made without departing from the spirit and scope of the disclosure. For example, the sensors can be made from different materials and have different configurations, as long as they maintain their flexibility, stretchability, and ability to accurately monitor and predict pressure ulcer development.

For example, in one additional variation of the current disclosure, the AI algorithm can be continually updated and improved based on ongoing research and development, allowing for more accurate and precise predictions of pressure ulcer development. The AI algorithm can also be adapted to incorporate new data sources and integrate with other medical monitoring systems, providing a more comprehensive view of a patient's overall health and well-being.

In addition, the stretchable sensor array system can be adapted for use in various other medical or non-medical applications that require continuous monitoring of pressure, temperature, humidity, or other factors. Examples of such applications include sports performance monitoring, sleep quality assessment, or monitoring the condition of items during transport or storage.

In some embodiments, the pressure-sensing mattress can be designed as a modular system, allowing users to add or remove sensors, customize the layout of the sensors, or adjust the sensitivity of the sensors based on their specific needs and preferences. This modularity enhances the personalization and adaptability of the pressure ulcer prevention system.

The system can also be configured to provide automated recommendations for repositioning, posture adjustments, or other interventions based on the data collected by the sensor array system and analyzed by the AI algorithms. This feature ensures that users receive personalized and timely guidance to reduce pressure ulcer risks.

The pressure ulcer prevention system can be integrated with mobile applications, smart home devices, or other digital platforms to provide users with real-time data, notifications, and recommendations, further enhancing the accessibility and usability of the system.

In some embodiments, the pressure-sensing mattress may have a built-in memory or data storage system, allowing users to track and analyze their pressure ulcer risk data over time. This feature can help users identify patterns or trends in their pressure ulcer risks, enabling more proactive and targeted interventions.

In other embodiments, the stretchable sensor array system can be integrated into specialized garments or wearable devices designed for specific body regions that are at a high risk of pressure ulcer development, such as heels, elbows, or sacral areas. These specialized garments or wearable devices can provide targeted monitoring and prevention of pressure ulcers in vulnerable areas.

While the present disclosure has been described with reference to specific embodiments, it will be understood by those skilled in the art that various changes may be made, and equivalents may be substituted without departing from the scope of the disclosure. In addition, many modifications may be made to adapt a particular situation or material to the teachings of the disclosure without departing from the scope thereof. Therefore, it is intended that the present disclosure is not limited to the particular embodiments disclosed, but that the disclosure will include all embodiments falling within the scope of the appended claims.

We claim:

1. A stretchable sensor array system, comprising a stretchable substrate, a sensor array comprising a plurality of sensors deposited on the substrate, and a computing device,
wherein each sensor comprises a conductive electrode that contains one or more carbonaceous materials and is configured to respond to a parameter selected from pressure, temperature, humidity, and chemical substances, and
each sensor is signally connected to the computing device and transmits signals representing the parameter to the computing device for storage and/or processing.

2. The stretchable sensor array system of claim 1, comprising a plurality of stretchable substrates, each having a sensor array deposited thereon.

3. The stretchable sensor array system of claim 1, wherein the substrate is composed of materials selected from the group consisting of polydimethylsiloxane (PDMS), polyurethane, silicone rubber, fluorinated elastomers, butadiene-based elastomers, isoprene-based elastomers, styrene-butadiene rubber, acrylonitrile-butadiene rubber, natural rubber, and synthetic rubbers such as neoprene, nitrile rubber, and ethylene propylene diene monomer (EPDM) rubber, or other suitable stretchable polymers.

4. A stretchable sensor array system, comprising the stretchable sensor array of claim 1 and a computing device, wherein each sensor in the sensor array is signally connected to the computing device and transmits signals representing the parameter to the computing device for storage and/or processing.

5. The stretchable sensor array system of claim 4, comprising a plurality of the stretchable sensor arrays.

6. A system for pressure ulcer prevention and monitoring, comprising:
a stretchable sensor array system according to claim 5, wherein one or more sensor arrays are affixed to a patient's skin or a device the patient is in a physical contact with; and
a patient or caregiver notification system operatively connected to the computing device, wherein the patient or caregiver notification system comprises at least one of an audible alarm, a visual indicator, and a tactile indicator.

7. The system of claim 4, wherein the computing device is a workstation or a handheld device, and a software program is installed on the computer device for receiving, processing, and/or displaying data.

8. The system of claim 4, further comprising a cloud-based platform for storing, processing, and analyzing data related to factors associated with pressure ulcer development, and for facilitating communication between the sensor array system and the patient or caregiver notification system.

9. A method for preventing and monitoring pressure ulcers in a patient using the stretchable sensor array system of claim 4, comprising the steps of:
applying one or more sensor arrays to a patient's skin or a device the patient is in a physical contact with;
detecting one or more parameters selected from pressure, temperature, humidity, and volatile organic compounds (VOC) using the one or more sensor array; and
transmitting sensor data to the computing device to be analyzed and to determine one or more conditions of the patient.

10. The method of claim 9, wherein the one or more conditions is selected from a duration of mobility or immobility of the patient, a temperature at or new a body part of the patient, a humidity at or near a body part of the patient, and VOCs emitted from a body part of the patient, and further comprises the step of notifying the patient and/or a caregiver when a condition arrives at or exceeds a predetermined threshold value.

11. The method of claim 9, wherein the device the patient is in contact with is a bandage, a garment, a cushion, a mattress, a chair, a wheelchair, and a mattress topper.

12. A training simulator, comprising the stretchable sensor array of claim 1, an object that a trainee interacts with, wherein the sensor array is affixed to the object.

13. The training simulator of claim 12, wherein the sensor array comprises a plurality of pressure sensors and configured to receive one or more palpations selected from light palpation, deep palpation, light ballottement, and deep ballottement and outputs data representative of the one or more palpations.

14. The training simulator of claim 12, wherein the object is a manikin, a garment, a vest, or a helmet.

15. The training simulator of claim 12, wherein the simulator is used for monitoring pressure in orthopedic maneuvers, assessing pressure during intravenous catheter insertion, providing feedback on compression depth in CPR training, simulating palpation during physical examination, preventing pressure injuries during patient positioning, simulating wound care, and evaluating pressure during obstetric simulations.

16. A method for palpation simulation using the training simulator of claim 12, comprising:
affixing the sensor array to the object, wherein the object is a manikin and the sensor array comprises a plurality of pressure sensors;
performing palpation on the sensor array and causing the sensor array to transmit sensor data to the computing device; and
processing the sensor data and displaying the processed data, wherein the processed data reflects pressure applied during the performance of palpation.

17. A method for making the stretchable sensor array of claim 1, comprising:
preparing a first mixture of a first elastomer and a first conductive nanomaterial,
depositing the first mixture to a stretchable substrate by inkjet printing, screen printing, aerosol jet printing, material jetting, and liquid deposition to form a plurality of first sensor elements; and
subjecting the plurality of sensor elements to surface treatment to form a plurality of first sensors.

18. The method of claim 17, further comprises:
preparing a second mixture of a second elastomer and a second conductive material, depositing the second mixture to the stretchable substrate by inkjet printing, screen printing, aerosol jet printing, material jetting, and liquid deposition to form a plurality of second sensor elements; and
subjecting the plurality of second sensor elements to surface treatment to form a plurality of second sensors.

19. The method of claim 17, wherein the plurality of first sensors is selected from pressure sensor, temperature sensor, humidity sensor, and VOC sensor.
